(19) United States
(12) Patent Application Publication
(10) Pub. No.: US 2014/0121656 A1
(43) Pub. Date: May 1, 2014

(54) NERVE AND SOFT TISSUE CUTTING DEVICE

(71) Applicant: WARSAW ORTHOPEDIC, INC., Warsaw, IN (US)

(72) Inventor: William F. McKay, Memphis, TN (US)

(73) Assignee: Warsaw Orthopedic, Inc., Warsaw, IN (US)

(21) Appl. No.: 13/662,052

(22) Filed: Oct. 26, 2012

(51) Int. Cl. A61B 18/18

(52) U.S. Cl. 606/33

(57) ABSTRACT

Cutting devices including a probe or needle useful for resecting nerve and soft tissue via a minimally invasive procedure to alleviate pain are provided. The cutting devices include an internal passage and a distal end that can have a tip and a groove positioned near the tip or at the tip at the distal end of the probe. The probe or needle can have an electrically insulated layer or coating adjacent to and exposing the groove which is configured to receive pulsed plasma mediated radio frequency discharges adapted for cutting nerve and/or soft tissue. The internal passage of the probe or needle can be configured to engage a vacuum for suction of the cut nerve and/or tissue, or separate passage for injection of fluids, sealants or cements. Methods for cutting nerve and/or soft tissue utilizing the cutting devices are also provided.

### FIELD

The present invention relates generally to devices and methods for cutting a material or substance. More specifically, the devices and methods are useful for resecting nerve and/or soft tissue via a minimally invasive procedure to alleviate pain.

### BACKGROUND

Standard methods of cutting tissue may include using a scalpel, scissors, and radio frequency energy. Electrosurgical procedures and techniques using radio frequency energy are currently used since they generally reduce patient bleeding and trauma associated with cutting operations. Additionally, electrosurgical ablation procedures, where tissue surfaces and volume may be reshaped, cannot be duplicated through other treatment modalities.

Minimally invasive procedures in nerve and/or soft tissue such as the spine or the breast, however, are difficult to perform using standard scissors and scalpel. Furthermore, in a closed environment, radio frequency current dissipates into the surrounding tissue causing a decreased ability to achieve a current at the cutting electrode of sufficiently high density to initiate a cut. To overcome this problem, high power settings are often required to initiate the cut which often is painful and increases thermal damage to the tissue whether using a standard or a custom electrosurgical generator.

Another problem associated with cutting tissue is the control of bleeding. Radio frequency energy controls bleeding by coagulating small blood vessels. Another method of controlling bleeding is through the use of heat. For example, some commercially available scalpels use direct heat to control bleeding. However, while the bleeding is generally controlled, the cutting of tissue is often slower than with radio frequency energy and the knife edge readily dulls. Other commercially available scalpels use ultrasonic energy generally at 50 kHz to heat the tissue so as to coagulate severed blood vessels but cut slower than a standard electrosurgical electrode and are costly as a custom ultrasonic generator is required.

A further disadvantage of using radio frequency energy is the generation of smoke. The smoke is malodorous and can contain airborne viral particles that may be infectious. Furthermore, the smoke often obscures visualization of the procedure. When the smoke becomes too dense, the procedure is delayed until the smoke is released through one of the trocar ports and after enough carbon dioxide gas has reinsufflated the abdominal cavity. This unnecessarily prolongs the operative time.

Radiofrequency (RF) energy is used in a wide range of surgical procedures because it provides efficient tissue resection and coagulation and relatively easy access to the target tissues through a portal or cannula. Conventional monopolar high frequency electrosurgical devices typically operate by creating a voltage difference between the active electrode and the target tissue, causing an electrical arc to form across the physical gap between the electrode and tissue. At the point of contact of the electric arcs with tissue, rapid tissue heating occurs due to high current density between the electrode and tissue. This high current density causes cellular fluids to rapidly vaporize into steam, thereby producing a “cutting effect” along the pathway of localized tissue heating. Thus, the tissue is parted along the pathway of evaporated cellular fluid, inducing undesirable collateral tissue damage in regions surrounding the target tissue site. This collateral tissue damage often causes indiscriminate destruction of tissue, resulting in the loss of the proper function of the tissue. In addition, the device does not remove any tissue directly, but rather depends on destroying a zone of tissue and allowing the body to eventually remove the destroyed tissue.

Present electrosurgical techniques used for tissue ablation may suffer from an inability to provide the ability for fine dissection of soft tissue. The distal end of electrosurgical devices is wide and flat, creating a relatively wide area of volumetric tissue removal and making fine dissections along tissue planes more difficult to achieve because of the lack of precision provided by the current tip geometries.

In addition, identification of the plane is more difficult because the large ablated area and overall size of the device tip obscures the physician's view of the surgical field. The inability to provide for fine dissection of soft tissue is a significant disadvantage in using electrosurgical techniques for tissue ablation, particularly in arthroscopic, otolaryngological, and spinal procedures.

Traditional monopolar RF systems can provide fine dissection capabilities of soft tissue, but may also cause a high level of collateral thermal damage. Further, these devices may suffer from an inability to control the depth of necrosis in the tissue being treated. The high heat intensity generated by these systems causes burning and charring of the surrounding tissue, leading to increased pain and slower recovery of the remaining tissue. Further, the desire for an electrosurgical device to provide for fine dissection of soft tissue may compromise the ability to provide consistent ablative cutting without significant collateral damage while allowing for concomitant hemostasis and good coagulation of the remaining tissue.

Another problem with currently available RF nerve ablation devices is that they attempt to destroy nerve tissue from a central location including the tip of the device and a 3-D spherical or cylindrical zone around it. As a result, the further away the resecting ability is from the central zone the less effective the nerve destruction. Consequently, often the nerve is not adequately ablated leading to continued pain symptoms.

Further, the health care practitioner may have difficulty positioning the tip of the device in the optimal location to get an optimal and consistent clinical result. This may also result in unwanted necrosis of adjacent tissue, which can lead to clinical adverse events including subsequent repair of the necrotic tissue.

Other devices such as mechanical rongures can be used to remove soft tissue. However, these devices require the insertion of relatively large cannulas that further complicate the surgical procedure and can cause nerve compression and pain with variable clinical efficacy.

Accordingly, there is a need for devices and methods to provide efficient severing or cutting of nerve and/or soft tissue that can be used during a minimally invasive procedure and/or during an open surgical procedure. Further, there is also a need for devices and methods that provide fine dissection capabilities of nerve and/or soft tissue. Devices and methods that do not cause a high level of collateral thermal damage and allow for the control of necrosis in the tissue being treated are also needed. Devices and methods that provide efficient, controlled and safe debulking of tissue would also be beneficial.

### SUMMARY

Cutting devices and methods are provided that allow resecting of the nerve and other soft tissue in a minimally invasive procedure. The cutting devices and methods provided allow the tip of the device to be easily positioned in an optimal location to obtain more efficient, better control, and safer resection and/or debulking of tissue with minimal unwanted destruction to adjacent nerve and soft tissue. In some embodiments, a device and method is provided that can debulk soft tissue that causes nerve compression and pain. The device and method has the ability to debulk tissue by passing the blade over the tissue as opposed to making several incisions with a scalpel and/or taking repeated bites with a mechanical ronguer instrument.

In some embodiments, the cutting devices and methods provided allow resecting nerves and other soft tissue via a minimally invasive procedure to alleviate pain. The cutting devices and methods disclosed herein comprise a probe having an internal passage and a distal end, the distal end having a tip and a groove positioned near the tip at the distal end of the probe. In various embodiments, the tip of the probe can be blunt. The probe comprises an electrically insulated layer or coating adjacent to and exposing the groove. The groove of the probe is configured to receive pulsed plasma mediated RF discharges adapted for cutting nerve and/or soft tissue. In some embodiments, the groove is an opening. The internal passage of the probe can be configured to engage a vacuum for suction of the cut nerve and/or soft tissue, and/or an additional channel for delivering fluid to the surgical site to wash out the area, facilitate suction of loose tissue fragments, and/or to cool the tissue.

In various embodiments, the cutting device comprises a needle or probe that is inserted at or near a target nerve or tissue, and once in position a plasma cutting blade is briefly activated as the needle or probe is physically manipulated into the nerve or tissue to be resected and/or debulked. The plasma cutting blade has the ability to cut through soft tissue with little or no biological effect on adjacent tissues while cauterizing blood vessels. The needle or probe has suction capability to remove resected tissue. The needle or probe, in some embodiments, can be equipped with navigation capability and/or with a pre-procedure CT (or MRI) so that the target nerve or soft tissue can be identified and accurately located during the resection procedure.

In some embodiments the hollow probe or needle has a pointed or blunt tip and the groove can be in the side of the distal tip of the needle. The edges of the groove are configured to receive pulse plasma mediated RF discharges and form a plasma cutting blade. Once the probe or needle having a plasma cutting blade is positioned over the nerve or soft tissue to be resected, the blade is briefly activated as the needle is physically manipulated into the nerve or soft tissue to be resected with a slight pulling back or pushing forward action to cut the nerve and/or soft tissue. Having the groove on the side of the blade provides for a more efficient, better control, and therefore safer method for “debulking” of tissue than current devices such as a scalpel or rongeurs. Thereafter, the resected tissue is removed by vacuum created suction available within or without the probe or needle.

In another embodiment, the probe or needle has the RF emitting groove in the side of the distal tip of the needle, but the plasma blade comprises a rotating cylinder or oscillating blade inside the probe or needle. Any resected tissue can also be removed by vacuum created suction available within or without the probe or needle.

In various embodiments, the probe or needle contains the RF emitting groove in the distal tip of the probe or needle. The plasma blade is inside the needle and comprises a rotating cutting blade that resects any tissue protruding into the groove as the probe or needle is manually pushed into it. Suction available within or without the probe or needle removes any resected tissue. In these embodiments the groove is closed by the inactivated plasma blade as the probe or needle is inserted into the desired location. However, once the desired location is reached, the groove opens and becomes activated with RF discharges and is ready for cutting.

In certain embodiments, the cutting device described herein further comprises a cutter within the probe or needle. The cutter comprises a rotating or oscillating blade around a shaft, the shaft extending parallel within the probe or needle for coupling the blade to a rotating or oscillating motion source. In various embodiments, the rotating blade contains a notch adapted to receive pulsed plasma mediated RF discharges proximate the groove and movable with respect to the groove in a distal direction while the notch is cutting nerve and/or tissue. As in other embodiments, the resected nerve and/or tissue are removed by vacuum created suction available within or without the probe or needle.

In certain embodiments, the rotating blade can have a regular or irregular polygon shape including a square, a rectangle, a circle, or an oval shape, the shape having smooth, beveled or ridged edges.

In other embodiments, it is contemplated that the oscillating blade is adapted to receive pulsed plasma mediated RF discharges proximate the groove and movable with respect to the groove in a distal direction while the oscillating blade is cutting nerve and/or tissue.

In yet other embodiments, the cutter further includes a cutter within the probe or needle, the cutter having a blade rotating around a shaft, the shaft extending parallel within the probe for coupling to a rotating motion source, for example a motor. The rotating blade is adapted to receive pulsed plasma mediated RF discharges proximate the groove while the groove is at the distal tip of the probe.

In certain embodiments, methods for resecting nerves and other soft tissue via a minimally invasive procedure to alleviate pain are also provided. Resection of the target nerve or soft tissue can eliminate and/or reduce pain symptoms. Specific clinical applications of the disclosed cutting instrument include resection of nerves causing discogenic back pain, leg pain, facet pain, resection of soft tissue causing stenosis pain symptoms, and many other orthopedic and oral maxillofacial pains.

In some embodiments, methods of resecting nerve and/or soft tissue include positioning a distal region of the probe of a cutting device adjacent a nerve or soft tissue to be cut. The probe of the cutting device has an internal passage and a distal end, where its distal end has a tip and a groove positioned near the tip. At its proximate end, the probe has a vacuum configured for suction of the cut nerve and/or tissue. The groove of the probe or needle can be configured to receive pulsed plasma mediated RF discharges adapted for cutting nerve and/or soft tissue. When the groove is moved over the nerve and/or soft tissue to be severed, the nerve and/or soft tissue are pulled back into the groove and ablated or cut by the pulsed plasma radio frequency discharges emanating from the groove. Thereafter, the vacuum at the proximate end of the probe is engaged for suction of the cut nerve and/or tissue.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

In part, other aspects, features, benefits and advantages of the embodiments will be apparent with regard to the following description, appended claims and accompanying drawings where:

FIG. 1 illustrates a cross-sectional view of a cutting device in accordance with one embodiment of the present disclosure;

FIG. 2 illustrates a cross-sectional view of the tip of the probe shown in FIG. 1 in accordance with one embodiment of the present disclosure;

FIG. 3 illustrates a cross-sectional view of a cutting device in accordance with another embodiment of the present disclosure;

FIG. 4 illustrates a perspective view of a rotating blade in accordance with another embodiment of the present disclosure;

FIG. 5 illustrates a perspective view of an oscillating blade in accordance with another embodiment of the present disclosure;

FIG. 6 illustrates a cross-sectional view of a cutting device in accordance with yet another embodiment of the present disclosure;

FIG. 7 illustrates a cross-sectional view of the tip of the probe shown in FIG. 6 in accordance with an embodiment of the present disclosure;

FIG. 8 illustrates a cross-sectional view of the tip of the probe shown in FIG. 6 in accordance with another embodiment of the present disclosure;

FIG. 9 illustrates a cross-sectional view of the tip of the probe shown in FIG. 6 in accordance with yet another embodiment of the present disclosure;

FIG. 10 illustrates a cross-sectional view of a cutting blade in accordance with yet another embodiment of the present disclosure;

FIG. 11 illustrates a side view of a cutting device in accordance with another embodiment of the present disclosure; and

FIG. 12 illustrates a side view of a tip of the probe shown in FIG. 11 in accordance with another embodiment of the present disclosure.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

Devices for efficient severing or cutting of a material or substance such as nerve and/or soft tissue suitable for use in open surgical and/or minimally invasive procedures are disclosed. The following description is presented to enable any person skilled in the art to make and use the present disclosure. Descriptions of specific embodiments and applications are provided only as examples and various modifications will be readily apparent to those skilled in the art.

The present disclosure may be understood more readily by reference to the following detailed description of the disclosure presented in connection with the accompanying drawings, which together form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure.

Definitions

As used in the specification and including the appended claims, the singular forms “a,” “an,” and “the” include the plural, and reference to a particular numerical value includes at least that particular value, unless the context clearly dictates otherwise.

Ranges may be expressed herein as from “about” or “approximately” one particular value and/or to “about” or “approximately” another particular value. When such a range is expressed, another embodiment includes from the one particular value and/or to the other particular value.

Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure.

For purposes of the description contained herein, with respect to components and movement of components described herein, “forward” or “distal” (and forms thereof) means forward, toward or in the direction of the forward, distal end of the probe portion of the device that is described herein, and “rearward” or “proximal” (and forms thereof) means rearward or away from the direction of the forward, distal end of the probe portion of the device that is described herein. However, it should be understood that these uses of these terms are for purposes of reference and orientation with respect to the description and drawings herein, and are not intended to limit the scope of the claims.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc. and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features.

For purposes of the description contained herein, “vacuum” means pressure within a space that is lower by any amount than atmospheric or ambient pressure, and although not exclusive of a condition of absolute vacuum defined by a complete absence within a space of air, fluid or other matter, the term as used herein is not meant to require or be limited to such a condition.

The headings below are not meant to limit the disclosure in any way; embodiments under any one heading may be used in conjunction with embodiments under any other heading.

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents that may be included within the invention as defined by the appended claims.

### Radiofrequency Ablation

Radiofrequency (RF) ablation devices have been available to surgeons to treat many medical conditions, for example, in the treatment of tumors in lung, liver, kidney, bone and other body organs. Pulsed RF has also been used for treatment of tumors, cardiac arrhythmias, chronic and post-operative pain, bone fracture and soft tissue wounds.

Medtronic Inc. is the owner of Peak™ plasma blade technology for tissue dissection surgical devices used in conjunction with Pulsar® generator to produce short plasma-mediated electrical discharges. The Pulsar® generator can supply pulsed waveforms that produce short plasma-mediated electrical discharges through a plasma blade.

Because the radiofrequency is provided in short on-and-off pulses with low duty cycle, and the blade is insulated, heat diffusion and associated heat damage to surrounding tissues is limited, resulting in less collateral damage and more precise tissue dissection. This technology is the subject of a variety of patents and patent applications including U.S. Pat. Nos. 6,135,998, 6,730,075, 6,780,178, 7,238,185, 7,357,802, 7,789,879 and 8,177,783 included herein by reference as if set forth in full.

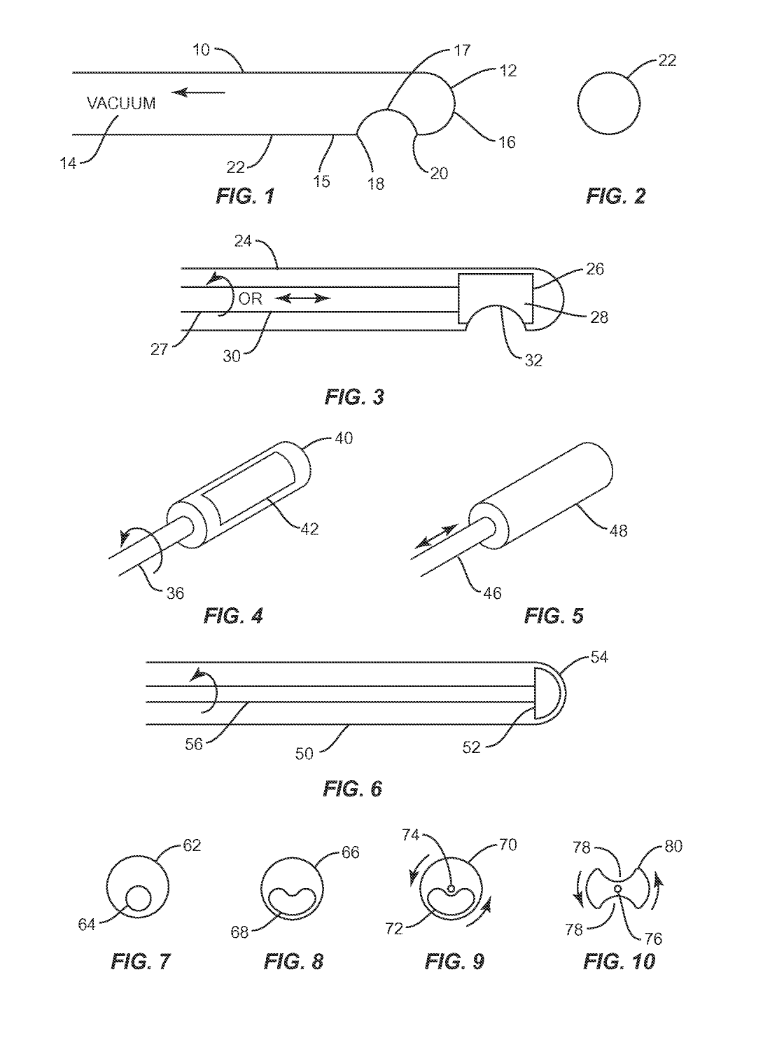

As illustrated in FIGS. 1 and 2, the present cutting device 10 comprises a probe 12 having a distal end 16, a tip 22 and a groove 17 positioned near the tip at the distal end 16 of probe 12. As shown in FIG. 2, tip 22 of probe 12 can be blunt.

In other embodiments, the tip of the probe can be round and/or somewhat pointed to allow for easy pushing through tissues. In some embodiments, probe 12 can be operatively connected to semi-steerable or navigational sources for easier guidance into tissues. In various embodiments, the navigational sources can be coupled with a pre-procedure such as for example, CT, MRI, PET scan, etc. so that the target nerve or soft tissue to be cut can be identified and accurately located during the procedure.

In various embodiments, probe 12 can be a hollow needle having a blunt tip 22 and a groove 17 positioned near the distal end of probe 12. The dimensions of the probe, among other things, will depend on the site that needs cutting. For example, the width of the epidural space is only about 3-5 mm for the thoracic region and about 5-7 mm for the lumbar region. Thus, the probe, in various embodiments, can be designed for these specific areas.

Some examples of lengths of the probe, may include, but are not limited to, from about 50 to 250 mm in length, for example, about 100 mm for epidural pediatric use, about 175 mm for a standard adult and about 225 mm for an obese adult patient. The thickness of the probe will also depend on the site of that needs cutting. In various embodiments, the wall thickness includes, but is not limited to, from about 0.05 to about 1.655 mm. The probe may be the widest or smallest diameter or a diameter in between for insertion into a human or animal body. The widest diameter is typically about 14 gauge, while the smallest diameter is about 25 gauge. In various embodiments the probe can be about 18 to about 22 gauge.

In some embodiments, the probe 12 has a blunt tip 22 that is centrally positioned, the surgeon or health practitioner can eliminate any difficulty in positioning the probe tip in the optimal location to get an optimal and consistent clinical result. The use of probe 12 or needle results in avoiding necrosis of adjacent tissue, which can lead to clinical adverse events that requires the tissue to undergo excessive repair itself after the procedure. In some embodiments, a central positioning of the tip 22 allows RF to be applied near the tip and avoid hemisphere spacing around the tip to avoid unwanted necrosis.

Groove 17 extends from edges 18 and 20 and is configured to receive pulsed plasma mediated RF discharges adapted for cutting nerve and/or soft tissue. In various embodiments, groove 17 has edges shaped as a regular or irregular polygon including arcuate, round, square, oblong, kidney shaped, crescent, or beveled shape with or without ridges.

Probe 12 includes an internal passage configured to engage a vacuum 14 to suction the resected nerve and/or soft tissue. Alternatively, an additional channel is possible for delivering fluid to the surgical site.

With further reference to FIG. 1, at its proximate end, probe 12 can be operatively connected to vacuum 14 for providing suction to resected nerve and/or tissue. Vacuum 14 may be used to transmit vacuum from a vacuum source (not shown) to a receiving aperture (not shown) connected to probe 12. Any suitable aspirator, cylindrical or otherwise, or other mechanism that creates vacuum upon the movement of an actuating member thereof, may be utilized as a vacuum source. Vacuum 14 can be in fluid communication with groove 17 for providing suction to remove cut nerve and/or soft tissue.

Groove 17 can have any shape allowing for nerve and/or soft tissue to be pulled back into the groove and ablated or resected with pulsed plasma radio frequency discharges. Groove 17 can have edges, each of which is shaped as a regular or irregular polygon including arcuate, round, square, oblong, kidney shaped, beveled shape with or without ridges. In some embodiments, groove 17 can be C-shaped and trap the tissue to be resected in the groove. By moving the probe or needle back and/or forth the RF will cut the tissue and the vacuum can be activated and the cut tissue can be removed.

In some embodiments, once the probe or needle is positioned over the nerve or soft tissue to be resected the blade can be briefly activated as the probe or needle is physically manipulated into the nerve or soft tissue to be resected and, with a slight pulling action, the nerve is pulled back and cut and the remaining resected tissue suctioned up by vacuum that can be engaged within the probe or needle. In another embodiment, groove 17 can be manipulated with a slight forward action over the nerve and/or soft tissue to be resected with the remaining cut tissue suctioned by vacuum 14.

With further reference to FIG. 1, not shown is an overall glass or other electric insulating layer covering most of the structure but leaving the C-shaped section of groove 17 exposed. Thus, there is no coating, insulating layer, or other material that prevents RF energy from leaving the probe or needle. In this way, RF energy is transmitted through the probe or needle and leaves out of groove 17 spanning 18 and 20 of FIG. 1, where tissue is cut by the RF energy of the groove 17. In some embodiments, the coating or insulating layer can be glass or ceramic having a thickness from about 0.005 to about 0.5 mm thick or from about 0.01 to about 0.2 mm thick. The insulation extends to the proximal portion of probe 12, but is not around groove 17.

The glass type insulation is typically applied by a conventional process of dipping each relevant component prior to assembly in liquid (molten) glass and then annealing the glass. As shown in FIG. 1, the coating or insulation layer does not cover the entire probe. Instead, from a location adjacent to groove 17, starting at about element 15 on probe or needle 12, groove 17 is uncovered by coating or insulation and is exposed through edges 18 to 20 to receive pulsed plasma mediated RF discharges adapted for cutting nerve and/or soft tissue.

Suitable material for probe or needle 12 can be for example, polyurethane, polyurea, polyether(amide), PEBA, thermoplastic elastomeric olefin, copolyester, and styrenic thermoplastic elastomer, steel, aluminum, stainless steel, titanium, nitinol, tungsten, molybdenum, metal alloys with high non-ferrous metal content and a low relative proportion of iron, carbon fiber, glass fiber, plastics, ceramics or a combination thereof.

In various embodiments, probe or needle may include radiographic markers to help indicate position on imaging procedures (e.g., CT scan, X-ray, fluoroscopy, PET scan, etc.). These may be disposed on or a portion of the probe or needle and include, but are not limited to, barium, calcium phosphate, and/or metal beads.

Probe 12 serves as a conduit for pulsed plasma mediated RF discharges. The actual nature of the applied electrical signals which are suitable to create the desired plasma effect is well known in the field. For instance, in one case the applied signal is an RF signal having a frequency in the range of 100 KHz to 10 MHz.

Typically this energy is applied in the form of bursts of pulses. Each burst typically has duration in the range of 10 microseconds to 1 millisecond. The individual pulses in each burst typically each have duration of 0.1 to 10 microseconds with an interval therebetween of 0.1 to 10 microseconds. The actual pulses are typically square waves and bi-phasic, that is alternating positive and negative amplitudes.

Generally the interval between pulses must be shorter than a lifetime of the plasma vapor cavity in order to maintain the cavity and the plasma regime during each pulse burst. In one embodiment, the bursts each are separated by duration of at least one millisecond.

In various embodiments, the time between the pulse bursts is sufficient so that the duty-cycle is relatively low as explained above. This minimizes the undesirable heating effects. However, in some embodiments, the provision of a cooling fluid reduces heating problems also. Typically, the plasma has a temperature greater than 100° C.

In some embodiments, the plasma blade formed around groove 17 is transiently activated with RF discharges after each push of the trigger that activates the RF so that excess tissue is not accidently removed. This unique design allows for an easy complete resection of pieces of nerve and/or soft tissue via a very small diameter, minimally invasive instrument with minimal disruptions of adjacent soft tissues.

Accordingly, as illustrated in FIGS. 1 and 2, probe 12 is an instrument that can be used for resecting nerves and other soft tissue via a minimally invasive procedure to alleviate pain. In various embodiments, probe 12 provides the additional flexibility resulting from utilizing a small diameter needle that can be pushed through tissue to a target nerve or tissue, and once in position groove 17, a plasma cutting blade, is briefly activated as probe 12 is physically manipulated into the nerve or tissue to be resected. The plasma cutting blade has the ability to easily cut through soft tissue with almost no biological effect on adjacent tissues while cauterizing blood vessels.

In other embodiments, as illustrated in FIGS. 3 and 4, probe 24 can include within its internal passage a cylinder 26 having a plasma blade 28 rotating around a shaft 27. The cutting edge of the plasma blade comprises groove 32, where tissue is trapped within the groove and can be cut and removed via vacuum. Shaft 27 extends parallel within probe 24 for coupling plasma blade 28 to a rotating motion source. The rotating motion source is adapted to rotate blade 28 at angles including 360°, 180°, 90° or 45° to a tissue plane to be cut. In this way, groove 32 can be positioned at its tissue cutting surface and cut tissue of the tissue plane.

Alternatively, the shaft can be moved in back and forth motion shown as 30, which will allow groove 32 to cut tissue. The shaft 27 will also be hollow and configured for creating a vacuum to remove tissue from the device once it is cut and to suction other material in the area.

As further illustrated in FIG. 4, rotating plasma blade 40 has a notch 42 adapted to receive pulsed plasma mediated radio frequency discharges proximate groove 32 shown in FIG. 3. With further reference to FIG. 3, rotating plasma blade 28 can be movable with respect to groove 32 in a distal direction while notch 42 rotating around shaft 36 is cutting nerve and/or tissue. Notch 42 of the rotating plasma blade 40 can have a regular or irregular polygon shape including a square, a rectangle, a circle, an oval, the shape having smooth, beveled or ridged edges. In some embodiments, plasma blade 40 can be a cylinder. In other embodiments, notch 42 of blade 40 can have a rectangular shape.

The cutting edge of the plasma blade 40 comprises notch 42, where tissue is trapped within the notch and can be cut and removed via vacuum. Shaft 36 extends parallel within the probe for coupling plasma blade to a rotating motion source. The rotating motion source is adapted to rotate blade 40 at angles including 360°, 180°, 90° or 45° to a tissue plane to be cut. In this way, notch 42 can be positioned at its tissue cutting surface and cut tissue of the tissue plane. The shaft 36 will also be hollow and configured for creating a vacuum to remove tissue from the device once it is cut and to suction other material in the area.

In yet other embodiments illustrated in FIG. 5, cylinder 48 can have a cutting edge on its front and the shaft 46 can move in back and forth motion. This type of cutting blade can replace the cutting blade in FIG. 3. The cylinder 48 can not only oscillate back and forth, it is also configured to receive pulsed plasma RF discharges out the front of cylinder 48 when moving forward and backward while cutting nerve and/or soft tissue. The shaft 46 will also be hollow and configured for creating a vacuum to remove tissue from the device once it is cut and to suction other material in the area.

As with cutting device 10 having a blunt tip 22 illustrated in FIGS. 1 and 2, probe 24 also has blunt tip 29 that is centrally positioned as shown in FIG. 3. As a result, the surgeon or health practitioner can eliminate any difficulty in positioning the probe tip in the optimal location to get an optimal and consistent clinical result. The use of probe 24 also results in avoiding necrosis of adjacent tissue, which can lead to clinical adverse events and requires these adjacent tissues to have to repair themselves. A central positioning of the tip 22 allows RF to be applied near the tip and avoid hemisphere spacing around the tip to avoid unwanted necrosis.

In other embodiments, as illustrated in FIGS. 6 to 9, hollow probe or needle 50 contains a rotating cutter 52, having a plasma blade 54 rotating around shaft 56. Shaft 56 extends parallel within probe 50 for coupling plasma blade 54 to a rotating or oscillating motion source. The rotating motion source is adapted to rotate blade 54 at angles including 360°, 180°, 90° or 45° relative to a tissue plane to cut.

In some embodiments, plasma blade 54 can be arcuate or have a rounded surface as illustrated in FIG. 6. In some embodiments plasma blade 54 can be semicircular in shape. In other embodiments, as illustrated in FIG. 10, the plasma blade 80 can have two arcuate opposing portions 78 forming four edges around pivot 76. The portions have tissue cutting edges. The plasma blades are adapted to receive pulsed plasma mediated RF discharges at a location proximate the groove and movable with respect to the groove in a distal direction while cutting nerve and/or tissue. The rotating motion source is adapted to rotate blade 80 at angles including 360°, 180°, 90° or 45° relative to a tissue plane to cut. The plasma blade can rotate in the device and allow tissue cutting around the circumference of the tissue and a vacuum applied within the hollow probe shaft will allow tissue and other material that has been cut to be suctioned out of the device and removed from the body.

In the embodiments illustrated in FIGS. 6 to 9 grooves 64, 68 and 72 can be located at the distal tips 62, 66, 70 of probe 50, respectively. Grooves 64, 68, and 72 can have edges shaped as a regular or irregular polygon including arcuate, round, square, oblong, oval, kidney shaped, beveled, or crescent shaped. The grooves can also include ridges or have no ridges. In various embodiments, grooves 64, 68 and 72 located at distal tips 62, 66 and 70 of probe 50 can be closed when the RF discharges of the plasma blade are inactive as the needle is inserted towards the desired location. Once the nerve or soft tissue to cut is reached, grooves 64, 68 and 72 open, the RF discharges become activated to cut the nerve or tissue protruding into each groove as the probe or needle is manually pushed into it.

In certain embodiments and with further reference to FIG. 9, the probe or needle can be provided with a tube or small channel 74 configured to deliver at the location of the severed nerve and/or soft tissue cement or polymer which can provide a physical barrier to prevent the temporary or permanent re-growth of nerve and/or soft tissue so that the pain symptoms do not return. This channel can be a separate channel from the vacuum channel and run parallel to it so that the device can cut and deliver a therapeutic material or barrier (e.g., polymer, cement, gel, etc.) to the area after cutting it.

As with cutting device 10 having a blunt tip 22 illustrated in FIGS. 1 and 2, and probe 24 having blunt tip 29 shown in FIG. 3, tips 62, 66, 70 of probe 50 illustrated in FIGS. 7, 8 and 9 are also blunt and centrally located. As a result, the surgeon or health practitioner can eliminate any difficulty in positioning the probe tip in the optimal location to get an optimal and consistent clinical result. The use of probe 50 also results in avoiding necrosis of adjacent tissue, which can lead to clinical adverse events and requires these adjacent tissues to have to repair themselves. A central positioning of the tips 62, 66 and 70 allows RF to be applied near the tip and avoid hemisphere spacing around the tip to avoid unwanted necrosis.

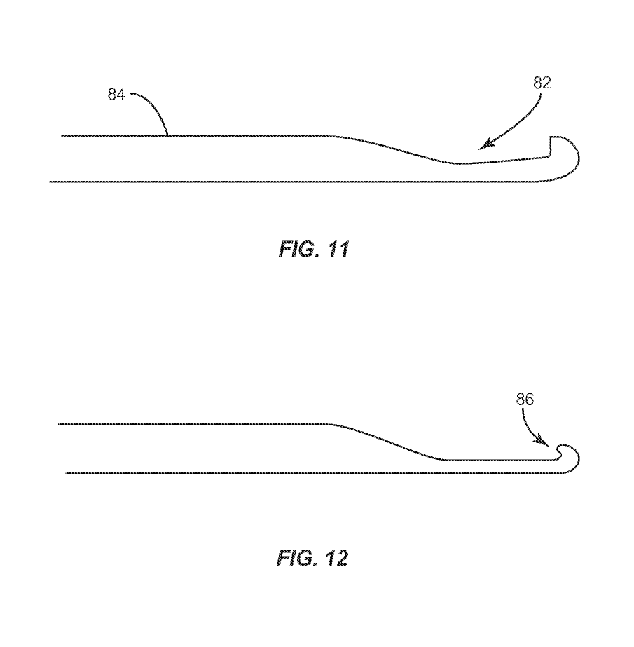

In other embodiments, as illustrated in FIGS. 11 and 12, the cutting device comprises a long groove 82. Groove 82 is scoop shaped and is configured for resecting and/or debulking tissue. In one embodiment, the cutting device is independent of a vacuum. In another embodiment, a portion 84 of the cutting device is a long tube and is collapsible and is configured for engagement with a vacuum. In other embodiments, the cutting device includes a tip 86 that has a decreased height relative to tips 62, 66 and 70.

### Methods for Cutting

The present disclosure also provides methods for cutting or resectioning nerve and/or soft tissue. The methods comprise positioning a distal region of a probe of a cutting device adjacent a nerve or soft tissue to be cut, the probe having an internal passage and a distal end, the distal end having a tip and a groove positioned near the tip at the distal end of the probe. The probe comprises an electrically insulated layer or coating adjacent to and exposing the groove, wherein the groove is adapted to receive pulsed plasma mediated radio frequency discharges adapted for cutting nerve and/or soft tissue, and the internal passage to the probe configured to engage a vacuum for suction of the cut nerve and/or tissue. The groove is subsequently moved over the nerve and/or soft tissue to be cut, and engages a vacuum within or without the probe to suction the cut nerve and/or soft tissue. In another embodiment, the cutting device defines a small channel configured for injection of irrigation fluid to the surgical site to wash out the surgical site, facilitate suction of loose tissue fragments, and/or to cool the tissue.

In other embodiments, the methods of the present disclosure further include delivering cement and/or a polymer through a small channel, such as small channel 74 in FIG. 9, for injection at the site of the nerve and/or soft tissue resection to provide a physically barrier at the location of the nerve resection to prevent temporary or permanent nerve regrowth, repair and return of the pain symptoms.

The barrier material utilized can be any suitable material effective to prevent or at least substantially inhibit the migration of substances that regrow tissue. Illustratively the barrier material can comprise a biodegradable synthetic polymer, in either flowable (and potentially hardenable) or non-flowable form. Illustratively, preferred barrier materials can have a first relatively flowable state during delivery and a second relatively less flowable state after implantation. For example, the barrier material may remain in an uncured, deformable, or otherwise configurable state during introduction, and rapidly cure, become harder or solidify after being introduced. Suitable materials that may be used for the barrier material include tissue sealants, adhesives, or implant materials made from natural or synthetic materials, including, for example, fibrin, albumin, collagen, elastin, silk and other proteins, polyethylene glycols (e.g. PEG gels), polyethylene oxide, cyanoacrylate, polylactic acid, polyglycolic acid, copolymers of polylactic acid and polyglycolic acid, polypropylene fumarate, tyrosine-based polycarbonate, ceramics, and combinations thereof. In some embodiments, the barrier material can be a cement.

In several embodiments, the methods disclosed herein include operatively coupling the probe to a source of navigational capability to allow easier pushing through the tissues. In various embodiments, the methods of cutting disclosed herein can include a pre-procedure step wherein the probe or needle can be coupled to a CT or MRI machine so that the target nerve and/or soft tissue to be cut can be identified and accurately located during the resection procedure.

The methods for cutting described hereinabove allow complete resection of the nerve avoiding the problems and partial effectiveness of current RF and cryoablation devices available in the art, and also allow for easier, more efficient, more complete, and safer removal of soft tissue that is causing stenosis pain symptoms.

As described above, the methods disclosed herein allow complete resection of the nerve avoiding the problems and partial effectiveness of current RF and cryoablation devices mentioned above, and also allow for easier, more efficient, more complete, and safer removal of soft tissue that is causing stenosis pain symptoms.

Specific clinical application of this instrument include resection of nerves causing discogenic back pain, leg pain, facet pain, resection of soft tissue causing stenosis pain symptoms, and many other orthopedic and oral maxillofacial pain. Many other painful conditions associated with arthroscopic, otolaryngological or spinal procedures could use the cutting devices and methods of using these cutting devices described herein.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. A cutting device comprising:
a probe having a distal end, the distal end having a tip and a groove positioned near the tip at the distal end of the probe, the probe having an electrically insulated layer or coating adjacent to and exposing the groove, wherein the groove is configured to receive pulsed plasma mediated radio frequency discharges adapted for cutting nerve and/or soft tissue.

2. A cutting device of claim 2, wherein the tip is blunt or pointed and wherein the groove has a shape configured to allow for nerve and/or soft tissue to protrude into the groove and ablated or cut with pulsed plasma radio frequency discharges and the cutting device comprises an internal passage configured to couple with a vacuum so as to remove cut or ablated tissue.

3. A cutting device of claim 3, wherein the groove has edges shaped as a regular or irregular polygon comprising arcuate, round, square, oblong, kidney shaped, beveled shape and/or the groove having ridges.

4. A cutting device of claim 1, wherein the groove is scoop shaped and is configured for resecting and/or debulking tissue.

5. A device of claim 2, further comprising a cutter within the probe, the cutter having a blade rotating around a shaft, the shaft extending parallel within the probe for coupling the blade to a rotating motion source, the rotating blade having a notch adapted to receive pulsed plasma mediated radio frequency discharges proximate the groove and movable with respect to the groove in a distal direction while the notch is cutting nerve and/or tissue.

6. A device of claim 5, herein the rotating motion source is adapted to rotate the blade at angles comprising 360°, 180°, 90° or 45° relative to a tissue plane.

7. A device of claim 5, wherein the notch of the rotating blade having a regular or irregular polygon shape comprising a square, a rectangle, a circle, an oval, the shape having smooth, beveled or ridged edges.

8. A device of claim 2, further comprising a cutter within the probe, the cutter having a blade oscillating back and forth around a shaft, the shaft extending parallel within the probe for coupling the blade to an oscillating motion source, the oscillating blade adapted to receive pulsed plasma mediated radio frequency discharges proximate the groove and movable with respect to the groove in a distal direction while cutting nerve and/or tissue.

9. A device of claim 2 further comprising a cutter within the probe, the cutter having a blade rotating around a shaft, the shaft extending parallel within the probe for coupling the blade to a rotating motion source, the blade adapted to receive pulsed plasma mediated radio frequency discharges proximate the groove and movable with respect to the groove in a distal direction while cutting nerve and/or tissue, wherein the groove is at the distal tip of the probe.

10. A device of claim 9, wherein the groove has edges shaped as a regular or irregular polygon comprising arcuate, round, square, oblong, oval, kidney shaped, beveled or having ridges.

11. A device of claim 9, wherein the blade is pivotally attached to the shaft, the blade having two opposed arcuate portions forming four cutting edges.

12. A device of claim 1, wherein the probe has the shape of a needle.

13. A device of claim 1, wherein the probe material comprises titanium, stainless steel, tungsten, molybdenum or alloys thereof.

14. A device of claim 1, wherein the insulating layer or coating comprises any dielectric material including glass or ceramic.

15. A device of claim 1, further comprising a channel proximate the distal tip of the probe for delivery of a biocompatible polymer at the location of nerve and/or soft tissue cutting to prevent nerve and/or soft tissue re-growth.

16. A device of claim 1, wherein (i) the probe is operatively coupled to a source of navigational capability or (ii) the probe is collapsible.

17. A device of claim 1, wherein the internal passage of the probe is configured to engage a vacuum for suction of the cut nerve and/or soft tissue.

18. A method of resecting nerve and/or soft tissue, comprising:
positioning a distal region of a probe of a cutting device adjacent a nerve or soft tissue to be cut, the probe having a distal end, the distal end having a tip and a groove positioned near the tip at the distal end of the probe, the probe having an electrically insulated layer or coating adjacent to and exposing the groove, wherein the groove is configured to receive pulsed plasma mediated radio frequency discharges adapted for cutting nerve and/or soft tissue; and moving the groove over the nerve and/or soft tissue for cutting.

19. A method of claim 17, further comprising delivering a polymer for injection at the nerve and/or tissue resection to prevent nerve and/or tissue regrowth and further comprising operatively coupling the probe to a source of navigational capability.

20. A method of claim 19, wherein the probe comprises an internal passage configured to engage a vacuum for suction of the cut nerve and/or soft tissue and the probe has the shape of a needle having a blunt or pointed tip.
